(19) United States
(12) Patent Application Publication
(10) Pub. No.: US 2011/0275925 A1
(43) Pub. Date: Nov. 10, 2011

(54) Method, System and Device for Acoustic and Photonic Tomography

(76) Inventor: Robert Leichner, Menlo Park, CA (US)

(21) Appl. No.: 13/141,666

(22) PCT Filed: Jan. 5, 2010
(86) PCT No.: PCT/US10/20152

§ 371 Date: Jun. 22, 2011

Related U.S. Application Data
(60) Provisional application No. 61/142,866, filed on Jan. 6, 2009.

(51) Int. Cl. A61B 6/00; A61B 5/05

(52) U.S. Cl. 600/407; 600/547

(57) ABSTRACT

Methods and devices for determining the distance between a location emitting an acoustic, photonic, or electromagnetic waveform and a location detecting the emitted waveform are provided. In certain applications the emitting and/or sensing locations may be selected from within a human, mammalian, or animal body. The subject methods and devices find use in a variety of different applications, including cardiac resynchronization therapy. In certain aspects, a bioelectric signal pathway carries a pulse from cardiac pacing pulse generator to deliver a pacing pulse to a heart while protecting a bioelectric measurement device from interference from the pacing pulse.

### CROSS-REFERENCE TO RELATED APPLICATIONS

This application claims the benefit of U.S. Provisional Patent Application No. 61/142,866, filed Jan. 6, 2009 and titled “Method, System and Device for Acoustic and Photonic Tomography”, incorporated by reference for all purposes in the Present Application.

### INTRODUCTION

Monitoring heart motion in the application of cardiac resynchronization therapy (hereinafter, “CRT”) is an area where accurate evaluations of organic tissue condition, position and motion are particularly valuable.

CRT is an important medical intervention for patients suffering from heart failure, such as congestive heart failure (CHF). CHF is generally characterized by a gradual decline in cardiac function punctuated by severe exacerbations that can lead eventually to death. CHF symptoms present due to the heart's inability to function sufficiently. It is estimated that over five million patients in the United States alone suffer from CHF.

CRT seeks to encourage a heart organ to exhibit a contraction time sequence that will most effectively produce maximal cardiac output with minimal total energy expenditure by the heart. The aim of resynchronization pacing is to induce the interventricular septum and the left ventricular free wall to contract at approximately the same time. Optimal heart contraction sequencing can be calculated by reference to hemodynamic parameters such as first time-derivative of the pressure waveform in the left ventricle, or “dP/dt”. The dP/dt parameter is a well-documented proxy for, and widely used indicator of, left ventricular contractility.

As currently delivered, CRT is effective in about one-half to two-thirds of patients implanted with a resynchronization device. In approximately one-third of these patients, this therapy provides a two-class improvement in patient symptoms as measured by the New York Heart Association scale. In about one-third of these patients, a one-class improvement in cardiovascular symptoms is accomplished. In the remaining third of patients, there is no improvement or, in a small minority, a deterioration in cardiac performance. This group of patients is referred to as non-responders.

CHF patients today are primarily managed on the basis of self-reported symptoms. In many cases, a patient's cardiovascular performance gradually deteriorates, with only mild subjective symptoms, until emergency admission to the hospital is required. The physician's ability to intervene early in the decompensation process—when cardiac performance is objectively declining but symptoms are not yet severe—is hampered by the lack of objective cardiac performance data characterizing the patient's condition.

The primarily symptomatic management of patients with or without heart failure in case of progressive ischemic heart disease is also problematic. Interventional cardiologists today have no reliable way of detecting an acute onset or worsening of cardiac ischemia when it is at an early, asymptomatic stage. If detected at this early stage, the ischemia is potentially reversible via a timely intervention. However, progressive akinesis, caused by stiffening of the cardiac muscle, is a hallmark of ischemia and is observable well before changes in the electrocardiogram (ECG) or in circulating cardiac enzymes.

Optimized CRT targets the cardiac wall segment point of maximal delay, and advances the heart movement timing to synchronize contraction with an earlier contracting region of the heart, e.g., the septum. However, the current placement technique for CRT devices is usually empiric. A physician might cannulate a vein that appears to be in the region described by the literature as most effective. A cardiac electrical stimulation element is then positioned, stimulation is carried out, and the lack of extra-cardiac stimulation, such as diaphragmatic pacing, is confirmed.

CRT optimization is currently attempted by a laborious and manual method of an ultrasonographer evaluating cardiac wall motion at different lead positions and different interventricular delay (hereinafter, “IVD”) settings. The ability to vary IVD settings enables a pacemaker to generate a pacing pulse that goes to the right ventricle versus the left ventricle at different timings relative to heart function. In addition, most pacemakers have the ability to vary the atrio-ventricular delay, i.e., the delay between stimulation of the atria and the ventricle or ventricles themselves. These timing control settings can be important in addition to the location of the left ventricular stimulating electrode itself in resynchronizing the patient.

An Implantable Cardioverter Defibrillator (“ICD”) is an implanted device that monitors the heart's electrical rhythm in order to detect and treat dangerous fast heart rhythms, ventricular tachycardia and ventricular fibrillation. It is advantageous to monitor the location and motion of a heart that is receiving pulse signals from a pacemaker or other electronic device. Yet these very pulse signals sent from a pacemaker, ICD or other electrical pulse generating devices can interfere with automated detection of signals and data that indicate locations or motion of elements of the heart. This data loss reduces a clinician's information about the actual functioning of a patient's heart.

Thus, the failure to reduce the loss of effective tomographic monitoring of the heart organ due to the degradation of parametric signals related to locations or motion of heart organ elements by electrical signal interference imposed by device-generated heart pulse signals reduce effective CRT.

### SUMMARY

Methods, systems, and devices for evaluating physical displacement between a signal emitting location and a signal arrival location are provided. In one aspect, a technique for monitoring of organic tissue location and motion, such as of a cardiac, thoracic, gastric, intestinal or other biological tissue or organ, e.g., a heart wall or a lung nodule, is provided.

In various aspects, an acoustic, photonic and/or electrical pulse or waveform may be generated proximate to the emitting location and the time of arrival of each pulse or waveform at the arrival location is measured or noted. The physical displacement between the emitting location and the displacement location is then calculated from a data set that includes the offset time between generation of at least two pulses or waveforms having different propagation rates, the arrival times of the at least two pulses or waveforms at the same arrival location, and the different rates of propagation of each of the at least two pulses or waveforms.

A calculation of displacement existing between the emitting location and the arrival location can be simplified and approximated under certain conditions. As acoustic waves propagate through organic tissue at 1540 meters per second and electromagnetic energy waves travel at a rate on the order of 10 to the eighth power meters per second through organic tissue, an approximation of an instantaneous arrival time of an electromagnetic pulse at an arrival location can simplify a displacement calculation in certain aspects of the present invention. In one exemplary variation of the present invention, a first energetic pulse comprised of electromagnetic energy and a second energetic pulse comprised of acoustic energy are generated at approximately the same moment from the emitting location. Under these conditions the measured difference in times of arrival of the two pulses can be approximated to be equal to the travel time of the acoustic pulse, as the electromagnetic pulse can be approximated to have arrived instantaneously at the arrival location after generation of the electromagnetic pulse at the emitting location, and the displacement between the emitting location and the arrival location can therefore be approximated to be equal to the product of a multiplication of (a.) the length of time measured at the arrival location between the arrival time of the electromagnetic pulse and the arrival time of the acoustic pulse; and (b.) the known rate of travel of the acoustic pulse within the medium disposed between the emitting location and the arrival location, e.g., organic tissue.

In certain still other aspects of the present invention a filter circuit is provided that reduces the loss of monitoring data imposed upon measuring equipment by the interference caused by a device generating a pulse or waveform. The filter circuit includes (1.) a low pass filter that reduces the high frequency component of a device-generated pulse; and (2.) a high pass filter that reduces the component of a measured signal that might include energy from another device-generated pulse. The pace generator may short out high frequency signals and the low pass filter prevents locking on the high frequency signals by pace circuits.

It is understood that the terms “pulse” and “waveform” are used synonymously in the present disclosure.

The subject methods and devices find use in a variety of different applications, including cardiac resynchronization therapy, kinesiology, monitoring of organic tissue, inspection of inorganic structures, and the monitoring of robotic or mechanical devices, equipment or elements.

### INCORPORATION BY REFERENCE

All publications, patents, and patent applications mentioned in this specification are herein incorporated by reference to the same extent as if each individual publication, patent, or patent application was specifically and individually indicated to be incorporated by reference. U.S. Pat. No. 5,983,126, entitled “Catheter Location System and Method”, issued on Nov. 9, 1999; United States Published Patent Application No. 2005/0038481, entitled “Evaluating Ventricular Synchrony Based On Phase Angle Between Sensor Signals”, published on Feb. 17, 2005; as well as U.S. application Ser. Nos. 11/249,152; 11/368,259; 11/555,178; 11/562,690; 11/615,815; 11/664,340; 11/731,786; 12/037,851; 11/219,305; 11/793,904; 11/917,992; 12/171,978; 11/909,786; are incorporated herein by reference in their entirety and for all purposes.

The publications discussed or mentioned herein are provided solely for their disclosure prior to the filing date of the present application. Nothing herein is to be construed as an admission that the present invention is not entitled to antedate such publication by virtue of prior invention. Furthermore, the dates of publication provided herein may differ from the actual publication dates which may need to be independently confirmed.

### BRIEF DESCRIPTION OF THE FIGURES

These, and further features of various aspects of the present invention, may be better understood with reference to the accompanying specification and drawings depicting various aspects of the present invention, in which:

FIGS. 1 to 3 provide depictions of various aspects of the subject invention configured for monitoring cardiac activity.

FIG. 4 is a schematic diagram of a second device comprising a controller, an emitter and a plurality of sensors.

FIG. 5 is a schematic diagram of the emitter of FIG. 4.

FIG. 6 is a schematic diagram of a representative first sensor of FIG. 4.

FIG. 7 is a flowchart of processing of the controller of FIG. 4.

FIG. 8 is a flowchart of computational processing and pulse generation of the emitter of FIGS. 4 and 5.

FIG. 9 is a flowchart of computational processing and sensing performance by a selected sensor of FIGS. 4 and 6.

FIG. 10 is a schematic drawing of a third device comprising the emitter of FIGS. 4 and 5 and the first sensor of FIGS. 4 and 6 are positioned external to the body of FIG. 1.

FIG. 11 is a schematic drawing of a fourth device comprising at least two sensors of FIGS. 4 and 6 are positioned on a robotic structure.

FIG. 12 is a schematic diagram of a variation of the first sensor of FIGS. 4 and 6, wherein the first sensor comprises a low frequency pass filter, a high frequency pass filter, a bioelectric signal sensor, and the cardiac electrode of FIGS. 5 and 6.

### DETAILED DESCRIPTION

It is to be understood that this invention is not limited to particular aspects of the present invention described as such and may, of course, vary. It is also to be understood that the terminology used herein is for the purpose of describing particular aspects only, and is not intended to be limiting, since the scope of the present invention will be limited only by the appended claims.

Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

A first method provides techniques and systems adaptable for use with tomographic techniques for evaluating motion of an organ or a living tissue of a living being. The living being may be an animal, or more particularly a “mammal” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In many applications, the subjects or patients will be humans.

The first method may be applied to living tissue and/or organs of living beings, such as a heart, a lung, a kidney, a limb, a section of dermis, a hand, a foot, a gut area, a digestive tissue, a bone, cartilage, and/or a muscle. According to the first method, movement or position of living tissue may be monitored at a cardiac location, such as at or proximate to a heart wall or an element of the diaphragm.

In the subject methods, a signal-sensing element may be stably associated with a tissue location of a living being, and a detection of an energy pulse or an energetic field may be performed by the signal-sending element. The detected energy pulse may be or comprise an electromagnetic energy component, an acoustic energy component and/or a photonic energy component. An energy field detected or detectable by the signal-sensing element may be an electromagnetic or electrostatic energy field.

The sensing-element is employed to evaluate movement or relative position of the tissue location. Also provided are systems, devices and related compositions for practicing the subject methods. The subject methods and devices find use in a variety of different applications, including cardiac resynchronization therapy, biometric signal analysis, kinesiology, monitoring, and other suitable applications known in the art.

Representative Methods

As summarized above, the first method provides methods of evaluating movement of a living tissue location. The subject methods may be characterized as electrical tomography enabling methods, wherein signals, i.e., energetic pulses or waveforms, produced by one or more emitting elements are detected by one or more sensing elements. It is understood that the terms “pulse” and “waveform”, when used herein in refer to a signal used to determine a displacement X, are synonymous terms.

While the methods may be viewed as electric tomography methods, such a characterization does not mean that the methods are necessarily employed to obtain a map of a given tissue location, such as a two-dimensional (2D) or three-dimensional (3D) map, but instead just that detections of energetic pulses or waveforms of a sensing element are used to evaluate or characterize a parameter, e.g., a tissue location or condition, etc., in some way. “Evaluating” is used herein to refer to any type of detecting, assessing or analyzing, and may be qualitative or quantitative. The tissue location evaluated in accordance with the various aspects is generally a defined location or portion of a body, i.e., subject, where in many cases it is a defined location or portion (i.e., domain or region) of a body structure, such as an organ, where in representative applications the body structure is an internal body structure, such as an internal organ, e.g., heart, kidney, stomach, lung, intestines, etc. The first method may be used in a variety of different kinds of animals, where the animals may be “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In many applications, the subjects or patients will be humans.

In many representative alternate applications of the first method, the tissue location is a cardiac location. As such and for ease of further description, the various aspects of the first method are now reviewed in terms of evaluating motion of a cardiac location. The cardiac location may be endocardial, epicardial, or a combination of both, as desired, and may be an atrial location, a ventricular location, or a combination of both. Where the tissue location is a cardiac location, in representative applications of the first method, the cardiac location is a heart wall location, e.g., a chamber wall, such as a ventricular wall, a septal wall, etc. Although the invention is now further described in terms of cardiac motion evaluation applications, the invention is not so limited, the invention being readily adaptable to evaluation of movement of a wide variety of mechanical systems, equipment control systems, robotics, as well as various tissue locations.

In practicing applications of the first method, an energy-emitting device is located relative to a human or a mammalian body, i.e., a “target body”. In certain applications, the energy-emitting device may emit a sound wave, an optical wave, and/or an electromagnetic pulse. The energy emitting device may be implantable such that the energy emitting device generates an acoustic, an electromagnetic, and/or a photonic pulse or waveform within the body, or alternately from locations outside of the body.

In the first method, following generation of the energetic pulse or waveform, as described above, a signal from energy emitting device is then detected by a sensing element at least twice over a duration of time to evaluate movement of the tissue location at which the sensing element is positioned. As the sensing element is stably associated with the tissue location, its movement is the same as the movement of the tissue location to which it is stably associated. The sensing element may be stably associated with the tissue location using any suitable means or method known in the art, such as by attaching the sensing element to the tissue location by using an attachment element, such as a hook, etc., by having the sensing element on a structure that compresses the sensing element against the tissue location such that the two are stably associated, etc. In certain applications, two or more different sensing elements are employed at different tissue locations. The number of different sensing elements that are employed in a given application may vary greatly, where in certain applications the number employed is two or more, such as three or more, four or more, five or more, eight or more, ten or more, etc.

The sensing element is or comprises, in representative applications of the first method, an acoustic energy sensing element, an electromagnetic sensing element, and/or a photonic sensing element. In these applications of the first method, the sensing element provides a value that indicates detection of an arrival of an acoustic, electromagnetic or photonic pulse or waveform to at least one controller. The at least one controller may reside within the sensing element, within a system comprising a pacemaker, and/or outside of the target body.

In certain applications, a single sensing element is employed. In such methods, evaluation may include monitoring movement of the tissue location over a given period of time.

In certain applications, two or more distinct sensing elements are employed to evaluate movement of two or more distinct tissue locations. In such applications, the methods may include evaluating movement of the two or more distinct locations relative to each other. In these applications, the evaluating may include assessing changes in distance between the two or more sensing elements, e.g., as is done in determining “Blood-Tissue Ratios”.

In certain applications, the subject methods include providing a system that includes an energy emitting device and one or more sensing elements, each emitting device and sensing element stably associated with a unique tissue location(s) of interest. This providing step may include either implanting one or more new elements into a body, or simply employing an already existing implanted system, e.g., a pacing system such as an adapter. This step, if employed, may be carried out using any suitable protocol known in the art or provided in the future such as a CRT therapy, the implantation and application of an ICD, and other suitable protocols known in the art, representative systems/devices and applications.

In one aspect of the first method, a system is employed that includes an energy-emitting device and a plurality of sensing elements. Each sensing element is configured to detect an energy pulse emitted by the energy emitting device, and each sensing element is stably associated with a cardiac location of interest, e.g., a heart wall, such as a ventricular wall, septal wall, etc., such that energetic pulse and waveform detections by the sensing element can be correlated with movement of the cardiac location of interest.

In representative applications of the first method, the system is comprised of the following main components: 1) three or more sensing elements; 2) at least one energy emitting device; and 3) a controller.

In certain alternate applications of the first method, the system includes pacing leads with a plurality of sensing electrodes placed around the heart, which provides a more comprehensive picture of the global and regional mechanical motion of the heart. With one or more sensing elements, artifacts such as breathing can be filtered out. Furthermore, multiple sensing elements of these alternate applications of the first method can provide 3D relative or absolute motion information by having one or more sensing elements configured to switch between the roles of an energy emitting device and a sensing element.

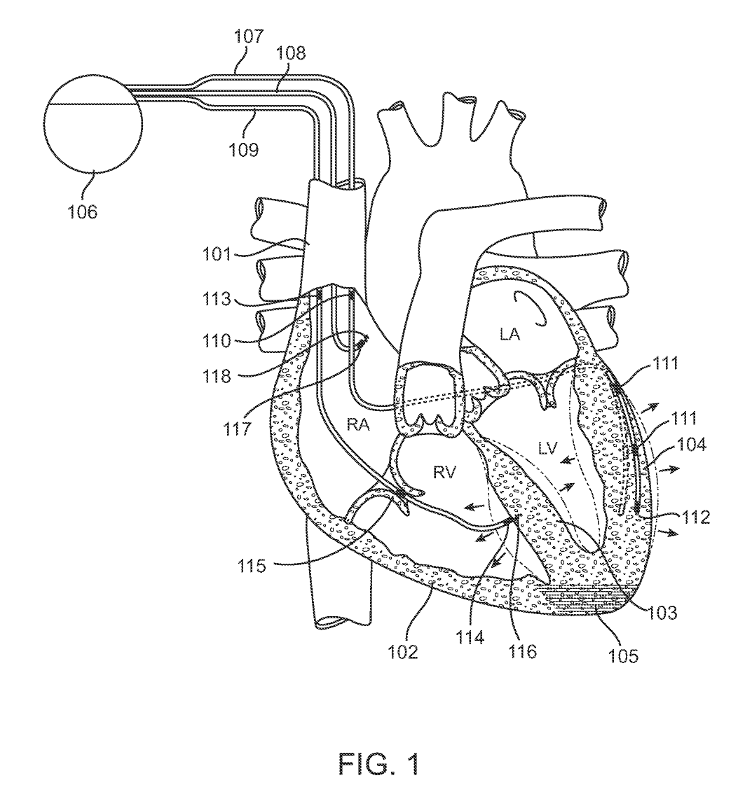

Intraventricular and interventricular mechanical dyssynchrony are useful synchrony indices used for optimizing CRT. Intraventricular dyssynchrony is defined as contractile timing dyssynchrony between the various left ventricular walls, in particular, the septal wall and the lateral wall. The intraventricular dyssynchrony can readily be measured by creating an electric field between two relatively unmoving electrodes (e.g. pacemaker can and electrode in basal region of heart) and measuring sensed voltage changes (e.g., resulting from contractile motion) in a sense electrode attached to the septal wall and a sense electrode in the left ventricle lateral wall (referenced to another electrode which may or may not be one of the driving electrodes). This electrode configuration is described below and shown in FIG. 1. The intraventricular dyssynchrony can be calculated by measuring the time interval between the contractile motion of the sense electrodes in the septal and lateral walls. Several time stamps of the contractile motion, such as onset of systolic contraction, peak systolic contraction, and peak velocity of contraction, can be used to make this calculation.

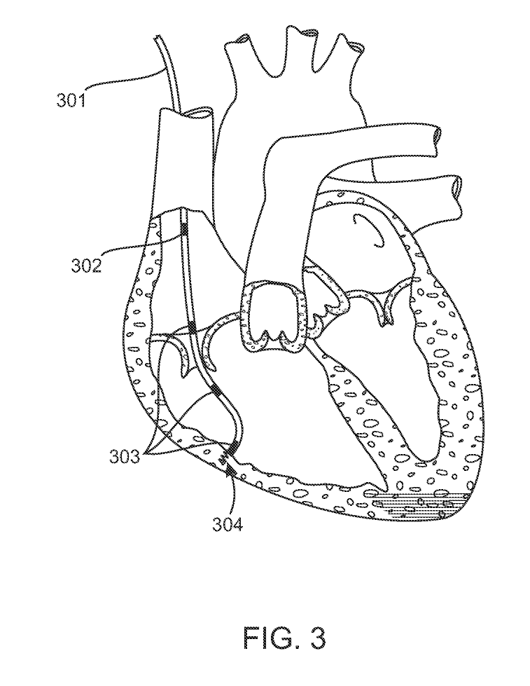

Interventricular mechanical dyssynchrony is defined as a global timing dyssynchrony between the right and left ventricle. The interventricular dyssynchrony can be determined by observing the relative movement between a selected device (e.g. pacemaker can), a first sensing element located at a septal wall, a second sensing elements attached to the right ventricle lateral walls, and a third sensing elements attached to the left ventricle lateral walls. These sensing element positions are described below and shown in FIGS. 1 and 3. Observing changes in locations of the left and right ventricle lateral wall sense electrodes provide global contractile timing information of the left and right ventricles. The interventricular dyssynchrony can be calculated by measuring the time interval between the global contractile motion of the right and left ventricle electrodes.

In certain applications, a plurality of energy emitting devices are present, each device configured to generate upon command at least one type of energetic pulse or waveform, e.g., acoustic, photonic, or electromagnetic energy, and one or more energy-emitting device may be positioned throughout the thorax, neck and abdomen, as well as external locations.

In certain applications of the system of the first method, relative timing and motion information is of greater importance than absolute position. In these applications, at least, significant movement of one or more sensing elements may be tolerated with minimal or even no real-time computation intended to compensate for this motion.

In certain applications, detection systems currently available for monitoring movement of a catheter inside a body are adapted for use in the subject methods.

Various systems are readily modified in order to track cardiac motion in accordance with the first method. In order to do so, these prior art systems are adapted to provide at least temporary if not permanent fixation of recording (i.e., sensing) elements in association with the region of the heart to be monitored.

Another application of the first method incorporates other physiologic sensors in order to improve the clinical utility of wall-motion data provided by the present invention. For example, an integrated pressure sensor could provide a self-optimizing cardiac resynchronization pacing system with an important verification means, since wall motion optimization in the face of declining systemic pressure would be an indication of improper pacing, component failure or other underlying physiologically deleterious condition (e.g., hemorrhagic shock). One or more pressure sensors could also provide important information used in the diagnosis of malignant arrhythmias requiring electrical intervention (e.g., ventricular fibrillation). Incorporation of other sensors is also envisioned.

In certain applications, the systems may include additional elements and features, such as a multiplexed system of the assignee corporation of the present application. This multiplexed system is described in part in currently pending patent applications U.S. patent application Ser. No. 10/764429 entitled “Method and Apparatus for Enhancing Cardiac Pacing”, U.S. patent application Ser. No. 10/764127 entitled “Methods and Systems for Measuring Cardiac Parameters”, and U.S. patent application Ser. No. 10/764125 entitled “Method and System for Remote Hemodynamic Monitoring”, all filed Jan. 23, 2004, U.S. patent application Ser. No. 10/734490 entitled “Method and System for Monitoring and Treating Hemodynamic Parameters” filed Dec. 11, 2003, U.S. Provisional Patent Application entitled “High Fatigue Life Semiconductor Electrodes” filed Dec. 22, 2004, and U.S. Provisional Patent Application entitled “Methods and Systems for Programming and Controlling a Cardiac Pacing Device” filed Dec. 23, 2004. Another related patent application is U.S. Provisional Patent Application entitled “Methods and Devices for Detecting Motion of Cardiac Tissue” filed Mar. 25, 2005. These patent applications are herein incorporated into the present application by reference in their entirety and for all purposes.

Certain other cardiac parameter sensing device are embodied in currently filed provisional patent applications; “One Wire Medical Monitoring and Treating Devices”, U.S. Provisional Patent Application No. 60/607280 filed Sep. 2, 2004, “Stable Micromachined Sensors” U.S. Provisional Patent Application 60/615117 filed Sep. 30, 2004, “Amplified Complaint Force Pressure Sensors” U.S. Provisional Patent Application No. 60/616706 filed Oct. 6, 2004, “Implantable Doppler Tomography System” U.S. Provisional Patent Application No. 60/617618 filed Oct. 8, 2004, “Cardiac Motion Characterization by Strain Measurement” U.S. Provisional Patent Application filed Dec. 20, 2004, and PCT Patent Application entitled “Implantable Pressure Sensors” filed Dec. 10, 2004, “Shaped Computer Chips with Electrodes for Medical Devices” U.S. Provisional Patent Application filed Feb. 22, 2005, Fiberoptic Cardiac Wall Motion Timer U.S. Provisional Patent Application 60/658445 filed Mar. 3, 2005, “Shaped Computer Chips with Electrodes for Medical Devices” U.S. Provisional Patent Application filed Mar. 3, 2005, U.S. Provisional Patent Application entitled “Cardiac Motion Detection Using Fiberoptic Strain Gauges” filed Mar. 31, 2005. These patent applications are incorporated by reference in their entirety and for all purposes.

Certain other alternate applications of the first method incorporate various display and software tools configured to coordinate multiple sources of sensor information. Examples of these can be seen in PCT application serial No. PCT/US2006/012246 titled “Automated Optimization of Multi-Electrode Pacing for Cardiac Resynchronization” and the priority applications thereof. These patent applications are incorporated by reference in their entirety and for all purposes.

The present invention permits use of intracorporeal electrodes for the added purposes described even if these electrodes are primarily intended for other applications (e.g., cardiac pacing). Some of the applications described employ permanently implanted devices, while others employ acute use. Cardiac wall motion is detected by fixing catheters in relation to the cardiac wall of interest. However, localization of the catheters themselves is an intrinsic attribute of the system. Therefore, catheter localization can also be accomplished. For example, one or more temporary electrophysiology catheter electrodes could be employed for additional energetic pulse or waveform sensing by using a permanently implantable device. Using the extracorporeal display system to communicate with the implantable component and incorporating the temporary sense electrodes, the system could provide non-fluoroscopic catheter localization. Additionally, if the temporary catheter were temporarily fixed in association with an otherwise unmonitored cardiac wall location, additional cardiac wall motion data would be generated in the course of an invasive cardiac study

In certain implantable applications of this invention wall motion, pressure and other physiologic data may optionally be recorded by an implantable computer. Such data can be periodically uploaded to computer systems and computer networks, including the Internet, for automated or manual analysis.

In CHF patients, the electromechanical delay of the left lateral ventricle increases due to the left branch bundle block and is one of the variables that CRT attempts to decrease. The electromechanical delay is defined as the time interval between the onset of the QRS waveform of the ECG and the initiation of systolic contraction. In certain applications of the first method, the electromechanical delay of the left lateral ventricle can be measured by observing the movement of a sensing element located in the left ventricle lateral wall. This sensing element is described below and shown in FIG. 1. The electromechanical delay is then determined by measuring the time interval between the onset of the QRS and the initiation of systolic motion.

In a fully implantable system, the sensing elements may be located such that the pacing timing parameters may be continuously optimized by the pacemaker. The pacemaker frequently determines the location of the sensing elements and parameters in order to minimize intraventricular dyssynchrony, interventricular dyssynchrony, or electromechanical delay of the left ventricle lateral wall in order to optimize CRT.

This cardiac wall motion sensing system of the first method can also be used during the placement procedure of the cardiac leads in order to optimize CRT. An external controller could be connected to the cardiac leads and skin-sensing element(s) during placement of the leads. The skin patch sensing element(s) act as the reference location until the pacemaker is connected to the leads. In this scenario, for example, the optimal left ventricle cardiac vein location for CRT is determined by acutely measuring intraventricular dyssynchrony.

FIG. 1 provides a cross-sectional view of a heart having a right ventricle lateral wall 102, an interventricular septal wall 103, a cardiac vein on the left ventricle lateral wall 104, an apex of the heart 105, a pacemaker 106, a left ventricle cardiac vein lead 107, a right atrium electrode lead 108, and a right ventricle electrode lead 109. The left ventricle cardiac vein lead 107 is comprised of a lead body and a most proximal electrode 110, a first distal electrode 111 and a second distal electrode 112. The first and second distal electrodes 111 and 112 are located in the left ventricle cardiac vein and provide regional contractile information about this region of the heart. Having multiple first and second distal electrodes 111 and 112 allows a choice of optimal electrode location for CRT. The most proximal electrode 110 may be located in the superior vena cava 101 in the base of the heart. This basal heart location is essentially unmoving and therefore can be used as one of the fixed reference points for the cardiac wall motion sensing system.

In one application, the left ventricle cardiac vein lead 107 is constructed with the standard and suitable materials known in the art for a cardiac lead such as silicone or polyurethane for the lead body, and MP35N for the coiled or stranded conductors connected to Pt-Ir (90% platinum, 10% iriudium) the first proximal electrode 110, the first distal electrode 111 and the second distal electrode 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of the left ventricle cardiac vein lead 107. The proximal end of the left ventricle cardiac vein electrode lead 107 connects to the pacemaker 106.

The left ventricle cardiac vein lead 107 may be placed in the heart using standard cardiac lead placement devices which include introducers, guide catheters, guide wires, and/or stylets. Briefly, an introducer is placed into the clavicle vein. A guide catheter is placed through the introducer and used to locate the coronary sinus in the right atrium of the heart. A guide wire is then used to locate a left ventricle cardiac vein. The left ventricle cardiac vein lead 107 is slid over the guide wire into the left ventricle lateral wall 104 and tested until an optimal location for CRT is found. Once implanted, the left ventricle cardiac vein lead 107 still allows for continuous readjustments of the optimal pacing electrode locations.

The right ventricle electrode lead 109 may be placed in the right ventricle of the heart. In this view, the right ventricle electrode lead 109 is provided with a proximal sensing element 113, a first sensing element 114 and a second sensing element 115. Each proximal sensing element 113 and first sensing element 114 and second sensing element 115 includes a pacing electrode capable of emitting a signal to encourage a desirable contraction of the heart.

The right ventricle electrode lead 109 may be placed in the heart in a procedure similar to the typical placement procedures for cardiac right ventricle leads. The right ventricle electrode lead 109 is placed in the heart using the standard cardiac lead devices which include introducers, guide catheters, guide wires, and/or stylets. The right ventricle electrode lead 109 may be inserted into the clavicle vein, thru the superior vena cava, through the right atrium and down into the right ventricle. The right ventricle electrode lead 109 may be positioned under fluoroscopy into the location the clinician has determined is clinically optimal and logistically practical for fixating the right ventricle electrode lead 109 and obtaining motion timing information for the cardiac feature area surrounding the attachment site.

Once the right ventricle electrode lead 109 is fixed on the septum, the right ventricle electrode lead 109 can be employed to provide timing data for the regional motion and/or deformation of the septum. The sensing element 115 includes a pacing electrode which is located more proximally along the right ventricle electrode lead 109, which provides timing data on the regional motions in those areas of the heart. By example, a sensing element 115 situated near the AV valve, which spans the right atrium in the right ventricle, provides timing data regarding the closing and opening of the valve. The proximal sensing element 113 is located in the superior vena cava 101 in the base of the heart. This basal heart location is essentially unmoving and therefore can be used as one of the fixed reference points for the cardiac wall motion sensing system.

The right ventricle electrode lead 109 may be fabricated of a soft flexible lead with the capacity to conform to the shape of the heart chamber. The right atrium electrode lead 108 is placed in the right atrium of the heart. The right atrium electrode lead 108 is used to both provide pacing and motion sensing of the right atrium.

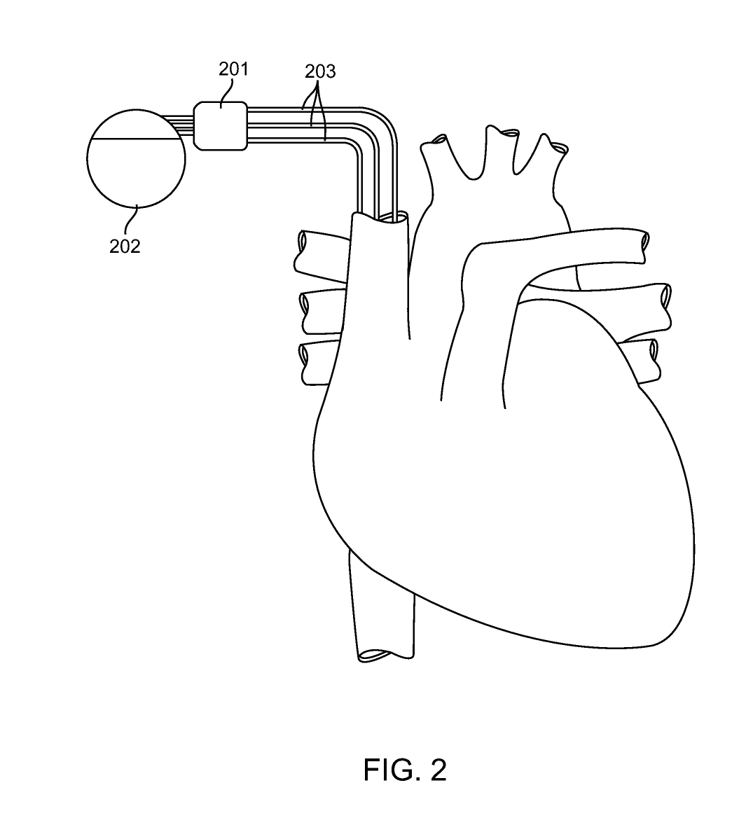

FIG. 2 provides a view of an additional of the device described in FIG. 1 with an add-on module 201 which is connected in series in between a pacemaker 202 and the additional electrode leads 203. Each electrode lead 203 includes multiple proximal sensing elements 113, wherein each proximal sensing element 113 includes a pacing lead for stimulating heart muscle contraction. The add-on module (i.e., adaptor) is comprised of a hermetically sealed housing which contains all the software, hardware, memory, wireless communication means, and battery necessary to run the cardiac wall motion sensing system. The housing is made of titanium and can be used as the reference electrode. On the proximal end, the add-on module 201 has lead type proximal connectors which can plug into the pacemaker header. On the distal end, the add-on module 201 provides connectors for additional electrode leads 203. One of the main advantages of the device of FIG. 2 is that the device of FIG. 2 may be configured for use with any commercial pacemaker 202. Even patients who already have a pacemaker 202 and lead system implanted can benefit from this add-on module 201. In an outpatient setting and using a local anesthetic a small incision is made expose the subcutaneously implanted pacemaker. The additional leads 203 are then disconnected from the pacemaker 202 and connected to the add-on module 201 which in turn is plugged into the pacemaker 202. The incision is then closed.

FIG. 3 provides a view of an alternate electrode lead 301 that includes a first alternate sensing element 302, a second alternate sensing element 303, and a third alternate sensing element 304 and is attached on the right ventricle lateral wall 304. Each alternate sensing element 302, 303, 304 may have a cardiac pacing lead (not shown) configured to stimulate heart muscle contraction.

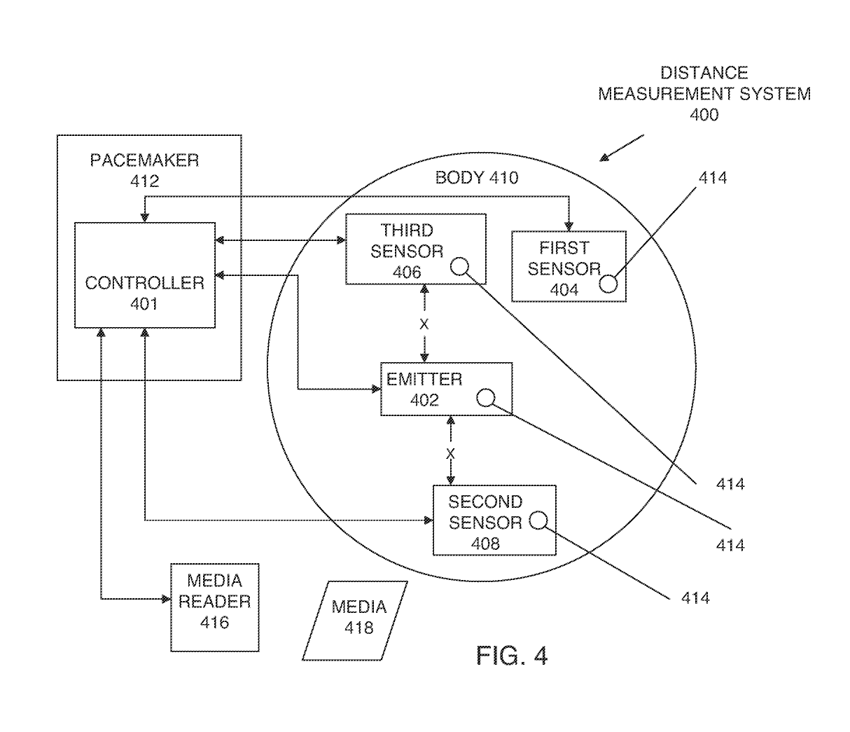

Referring now generally to the Figures and particularly to FIG. 4, FIG. 4 is a schematic diagram of a distance measurement system 400, hereinafter “second device” 400. In the second device a controller 401 is bi-directionally communicatively coupled with an energy emitting device (emitter) 402, a first sensing element (sensor) 404, a second sensing element (sensor) 406, and a third sensing element (sensor) 408. The energy emitting device 402 (or “emitter” 402) is configured to transmit both an electromagnetic pulse and an acoustic energy pulse. The first sensing element 404 (or “first sensor” 404), the second sensing element 406 (or “second sensor” 406) and the third sensing element 408 (or “third sensor” 408) are configured to detect both an electromagnetic pulse as generated by the emitter 402 and an acoustic energy pulse as also generated by the emitter 402. The emitter 402, first sensor 404, the second sensor 406 and the third sensor 408 are located within a human, mammalian, or animal body 410.

It is understood that configuration of the emitter 402, the first sensor 404, the second sensor 406 and the third sensor 408 may be similar in that the emitter 402 may be additionally configured to detect electromagnetic pulses and/or acoustic energy pulses, and the first sensor 404, the second sensor 406 and/or the third sensor 408 may be additionally configured to emit electromagnetic pulses and/or acoustic energy pulses. It is further understood that the controller 401 may comprise, or be comprised within, a pacemaker 412, and the emitter 402, first sensor 404, and/or second sensor 406 may each be separately configured with a muscle stimulation electrode 414.

The controller 401 may further comprise a media reader 416 that is selected and configured to read machine-executable, software encoded instructions from a computer-readable medium (media) 418.

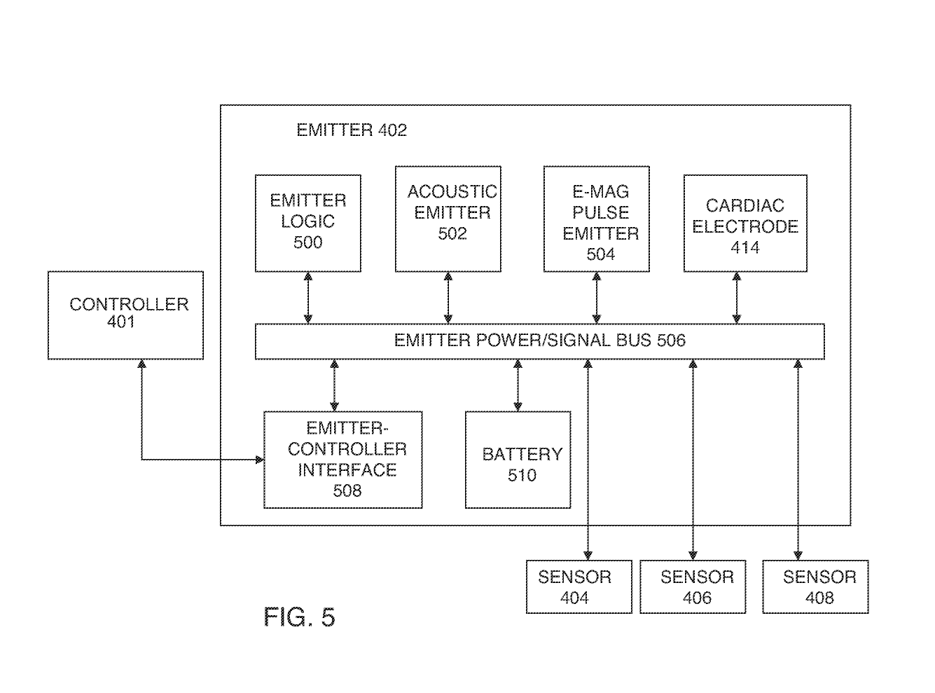

Referring now generally to the Figures and particularly to FIG. 5, FIG. 5 is a schematic diagram of the emitter 402 of the second version of FIG. 4. The emitter 402 includes an emitter logic 500, an acoustic pulse emitter 502, an electromagnetic pulse emitter 504, and a cardiac electrode 414. The emitter logic 500 is bi-directionally communicatively coupled with an emitter power and signal bus 506 (herein after “emitter bus” 506). The emitter bus 506 additionally bi-directionally communicatively couples the emitter logic 500, the acoustic pulse emitter 502, the electromagnetic pulse emitter 504, the cardiac electrode 414, and an emitter-controller interface 508. The emitter 402 may also optionally include an emitter battery 510 that provides electric power to the elements 414, 500-508 of the emitter 402, as well as optionally to the first sensors 404, the second sensor 406, and the third sensor 408.

In certain alternate applications of the second version, the first sensor 404, the second sensor, 406 and the third sensor 408 are bi-directionally communicatively coupled with the emitter bus 506, whereby the emitter logic 500 can communicate with the first sensor 404, the second sensor, 406 and the third sensor 408 via the emitter bus 506. The emitter 402 may emit a series of separate energetic pulses and the emitter logic 500 may direct each of the first sensor 404, the second sensor, 406 and the third sensor 408 to report a detection of particular energetic pulses within the series, whereby detection of each individual energetic pulse is reported by one and only of the first sensor 404, the second sensor, 406 and the third sensor 408.

In certain yet alternate applications of the present invention the emitter battery 510 may include a radio frequency identification circuit that accepts electrical energy from radio-wave transmissions and provides the received radio-wave transmitted electrical energy to the emitter 402, whereby the emitter 402 and/or the first sensor 404, the second sensor, 406 and the third sensor 408 are energized by conventional RFID technology.

The emitter-controller interface 508 is bi-directionally communicatively coupled with the controller 401 and provides a communication and electrical power pathway for electrical power, commands, data and status information between the controller 401 and the emitter logic 500 via the emitter bus 506. The emitter-controller interface 508 may, in certain alternate applications of the second version, provide a communication and electrical power pathway for electrical power, commands, data and status information between the controller 401 and other elements 500-510 of the emitter 402 and the first sensor 404, the second sensor, 406 and the third sensor 408, via the emitter bus 506.

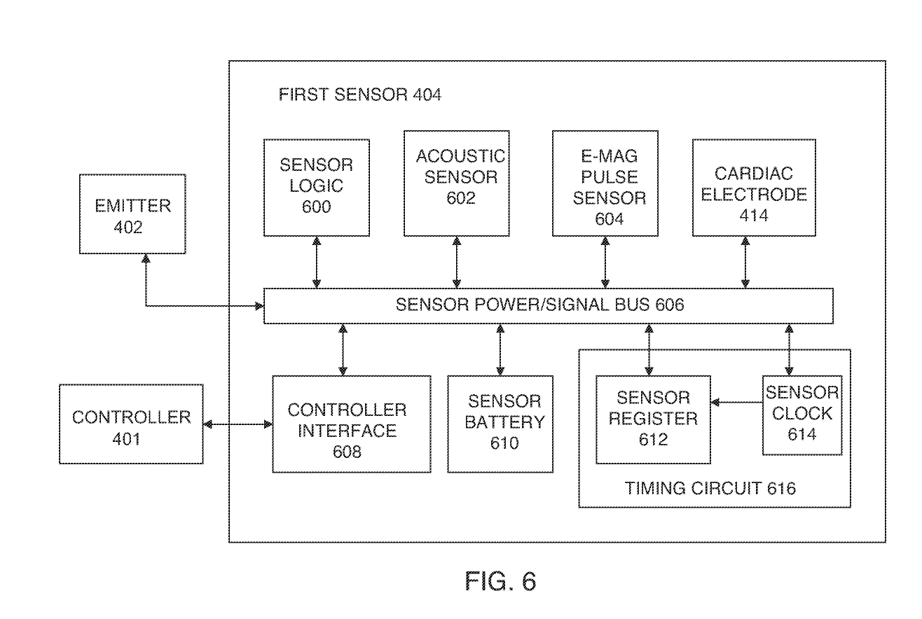

Referring now generally to the Figures and particularly to FIG. 6, FIG. 6 is a schematic diagram of a representative first sensor 404 of the second version of FIG. 4. It is understood that each and every element and aspect of the first sensor 404 may be comprised within one or more additional sensors 406 and 408 of the second version of FIG. 3.

The first sensor 404 includes sensor logic 600, an acoustic pulse sensor 602, an electromagnetic pulse sensor 604, and an optional cardiac electrode 414. The sensor logic 600 is bi-directionally communicatively coupled with a sensor power and signal bus 606 (herein after “sensor bus” 606). The sensor bus 606 additionally bi-directionally couples the sensor logic 600, the acoustic pulse sensor 602, the electromagnetic pulse sensor 604, the cardiac electrode 414, and a sensor-controller interface 608, an optional sensor battery 610, a sensor register 612 and a sensor clock 614. The optional sensor battery 610 may provide electric power to the elements 414, 600-614 of the first sensor 404.

In certain alternate applications of the second version, the first sensor 404 is communicatively coupled with the emitter 402, whereby the sensor logic 600 can communicate with the emitter 402 via the sensor bus 606. In certain yet alternate applications of the present invention the sensor battery 610 may include a radio frequency identification circuit that accepts electrical energy from radio-wave transmissions and provides the received radio-wave transmitted electrical energy to the first sensor 404, whereby the first sensor 404 is energized by, conventional RFID technology or other technologies.

The sensor-controller interface 608 is bi-directionally communicatively coupled with the controller 401 and provides a communication and electrical power pathway for electrical power, commands, data and status information between the first sensor 404 and the sensor logic 600 via the sensor bus 606. The sensor-controller interface 608 may, in certain alternate applications of the second version, provide a communication and electrical power pathway for electrical power, commands, data and status information between the first sensor 404 and other elements 600-614 of the first sensor 404.

The sensor register 612 and the sensor clock 614 comprise a timing circuit 616 configured to determine relative times and measure periods of time between times and/or pulse detections. The sensor register 612 is coupled with the sensor clock 614 and may be initialized by a command received via the sensor bus 606 from the controller 401, the emitter 402, and/or the sensor logic 600. Clock pulses from the sensor clock 614 increment the value of the sensor register 612 and thereby allow the register sensor 612 to maintain and provide a value proportional to a length of time that has passed since a most recent initialization of the sensor register 612.

The distances separating the emitter 402 and each of the first sensor 404, the second sensor, 406 and the third sensor 408 vary over time as the respective physiologic location(s) associated with the sensor(s), e.g., body tissue, organs, etc., move internally. The determination of a distance between the emitter 402 and a selected sensor 404, 406 and 408 existing at a particular moment can be determined by the aspects of the present invention by sending two different types of energy pulses from the emitter 402 to a selected sensor 404, 406 and 408 when each pulse has a different and known rate of propagation through the body 410 and the times at which each pulse was generated by the emitter 402.

In a general case of a first alternate implementation of the second version, the controller 401 directs the emitter 402 to generate an electromagnetic pulse at a time TGE, and to also generate an acoustic pulse at a time TGA. The controller 401 further directs the first sensor 404 to initialize the sensor register 612 to a zero reference time value V0 upon detection of the electromagnetic pulse by the electromagnetic pulse sensor 604. The instantaneous value of the sensor register 612 is then incremented upon receipt of each clock pulse from the sensor clock 614 by the sensor register 612. The first sensor 404 continues to increment the sensor register 612 upon each clock pulse of the sensor clock 614 until the first sensor controller 600 detects an arrival at the acoustic sensor 602 of the acoustic pulse sent from the emitter 402. The sensor logic 600 then reads an instantaneous second value VX of the sensor register 612 that indicates the number of clock pulses that the sensor register 612 received between (a.) the time of detection of the electromagnetic pulse by the electromagnetic sensor 604; and (b.) the time of detection of the acoustic pulse by the acoustic sensor 602.

The sensor logic 600 then subtracts the initialization zero value V0 from the second value VX, and converts the resulting clock cycle count to a real time value TD, where the real time value is a time period observed by the first sensor 404 between the (a.) the time of detection of the electromagnetic pulse by the electromagnetic sensor 604; and (b.) the time of detection of the acoustic pulse by the acoustic sensor 602. The first sensor 404 then transmits the real time value TD, or “time delay value” TD to the controller 401.

It is understood that the controller 401 may direct the emitter 402 to sequentially send paired instances of a particular electromagnetic pulse and a particular acoustic pulse for sequential detection by a specific sensor 404, 406 and 408, wherein each separate pair of pulses is used by a specific sensor 404, 406, and 408 to determine a specific real time delay value.

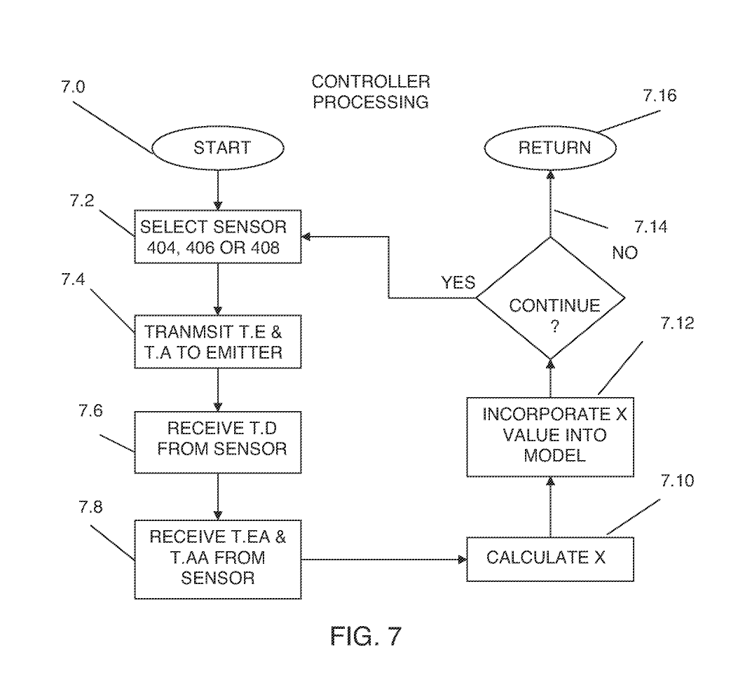

Referring now generally to the Figures and particularly to FIG. 7, FIG. 7 is a flowchart of processing of the controller 401 of FIG. 4. In step 7.2 the controller 401 determines which of the first sensor 404, the second sensor, 406 and the third sensor 408 is selected to examine for relative position to the emitter 402. The controller 401 transmits to the emitter 402 a first time T.E to emit an electromagnetic pulse and a second time T.A to emit an acoustic pulse T.A in step 7.4. The first time value T.E and the second time value T.A are each expressed in terms of relative time displacement from a time zero TZ. The time zero TZ may optionally be transmitted to the emitter 402 and/or the first sensor 404, the second sensor, 406 and the third sensor 408 selected in step 7.2. Preferably, the controller directs the emitter 402 to emit an electromagnetic pulse and an acoustic pulse at the same moment, i.e., where the first time T.E equals the second time value T.A. In step 7.6 the controller receives a delta time value T.D from the first sensor 404, the second sensor, 406 and the third sensor 408 selected in step 7.2, wherein the delta time value T.D is the time measured by the selected first sensor 404, the second sensor, 406 and the third sensor 408 of step 7.2 between detection by the selected first sensor 404, the second sensor, 406 and the third sensor 408 of the arrival of the electromagnetic pulse generated by the emitter 402 and the acoustic pulse generated by the emitter 402. In optional step 7.8 the controller receives from the selected first sensor 404, the second sensor, 406 and the third sensor 408 of step 7.2 both a first e-mag pulse detection time value T.EA and a second acoustic pulse detection time value T.AA. The first e-mag pulse detection time value T.EA is a measure of time passing between the time zero TZ that the selected first sensor 404, the second sensor, 406 and the third sensor 408 of step 7.6 detected an electromagnetic pulse sent from the emitter 402, and the second acoustic pulse detection time value T.AA is a measure of time passing between the time zero TZ that the selected first sensor 404, the second sensor, 406 and the third sensor 408 of step 7.6 detected an acoustic pulse sent from the emitter 402. The time delay value T.D is equal to the difference between the first e-mag pulse detection time value T.EA and the acoustic pulse detection time value T.AA.

The controller 401 calculates in step 7.10 the instantaneous distance X between the emitter 400 and the first sensor 404, the second sensor, 406 or the third sensor 408 selected in step 7.2. The controller bases this calculation of X on (a.) a pre-specified coefficient of propagation CE for the electromagnetic pulse generated by the emitter 402, (b.) a pre-specified coefficient of propagation CA for the acoustic pulse generated by the emitter 402, (c.) first time value T.E; (d.) the second time value T.A; and (e.) the delta time value T.D from the first sensor 404, the second sensor, 406 or the third sensor 408 selected in step 7.2.

The controller 401 may then proceed from step 7.10 to step 7.12 and to incorporate the value of X calculated in step 7.10 into a dynamically maintained and monitored model of the motion, position and condition of the body 410. The controller 401 proceeds from step 7.12 to step 7.14 and to determine whether to continue to measure the instantaneous displacement values of X between the emitter 402 and the first sensor 404, the second sensor, 406 and the third sensor 408. The controller 401 thus determines in step 7.14 whether to proceed back to step 7.2 and to direct another measurement of a displacement value X, or from step 7.14 to step 7.16 to perform alternate computational operations.

The controller 401 may derive a three-dimensional model of relative internal movements of the body 410 from calculated instantaneous displacement values of X between the emitter 402 and the first sensor 404, the second sensor 406 and the third sensor 408, wherein the relative position of the emitter 402 and each of the first sensor 404, the second sensor, 406 and the third sensor 408.

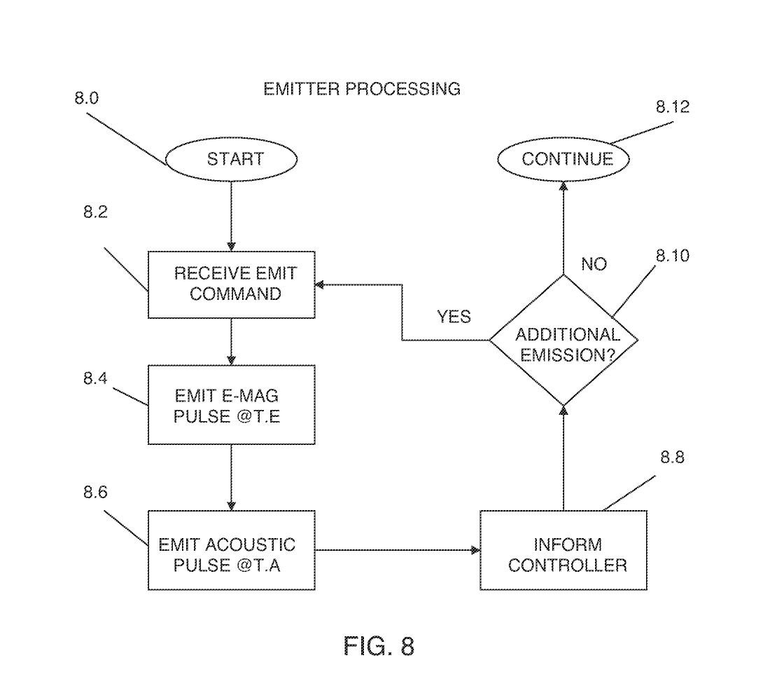

Referring now generally to the Figures and particularly to FIG. 8, FIG. 8 is a flowchart of computational processing and pulse generation of the emitter 402 of FIGS. 4 and 5. The emitter 402 receives a command to emit an electromagnetic pulse and an acoustic pulse from the controller 401 in step 7.2, the emit command specifying both a first time value T.E to generate an electromagnetic pulse and a second time value T.A for generating an acoustic pulse. Preferably, the first time value T.E is identical to the second time value T.A and an electromagnetic pulse and an acoustic pulse are simultaneously generated by the emitter 402, wherein steps 8.4 and 8.6 are executed simultaneously. In optional step 8.8, the emitter 402 informs the controller 401 that the electromagnetic pulse and the acoustic pulse have been generated.

The emitter 402 proceeds from step 8.8 to step 8.10 and to determine whether to continue to accept more commands from the controller 401 to generate pulses to support the measurement of instantaneous displacement values of X between the emitter 402 and the first sensor 404, the second sensor, 406 and the third sensor 408. The emitter 402 thus determines in step 8.10 whether to proceed back to step 8.2 and to accept another emit command from the controller 401, or from step 8.10 to step 8.12 and to perform alternate operations.

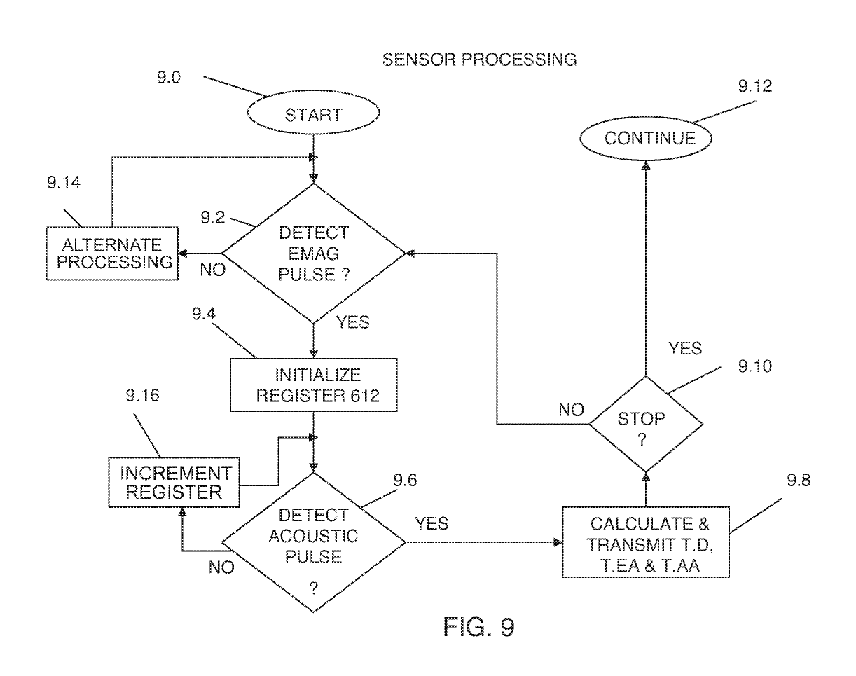

Referring now generally to the Figures and particularly to FIG. 9, FIG. 9 is a flowchart of computational processing and sensing performance by a selected first sensor 404, the second sensor, 406 and the third sensor 408 of FIGS. 4 and 6. For the purpose of clarity, the steps of the process of FIG. 9 is discussed regarding the first sensor 404, with the understanding that the discussion herein regarding the process of FIG. 9 may also be sequentially or simultaneously applicable to the second sensor 406, the third sensor 408, and additional sensors.

The first sensor 404 determines in step 9.2 whether an electromagnetic pulse has been detected, and proceeds on to initialize the sensor register 612 in step 9.4 when an electromagnetic pulse has been detected. The first sensor 404 proceeds from step 9.4 to step 9.6 and to determine whether an acoustic pulse has been detected, and proceeds on to step 9.8 when an acoustic pulse has been detected. The first sensor logic 600 then reads the sensor register 612 in step 9.8, then calculates the time delay value T.D therefrom (and optionally the time of arrival T.EA of the electromagnetic pulse and the time of arrival T.AA of the acoustic pulse, both arrival time values T.EA and T.AA expressed in terms of time displacement from the time zero TZ), and transmits the time delay value T.D (and optionally the pulse arrival time values T.AA and T.EA) to the controller 401 in step 9.8

The first sensor 404 proceeds from step 9.8 to step 9.10 and to determine whether to continue observing for additional occurrences of energetic pulses to support the measurement of instantaneous displacement values of X between the emitter 402 and the first sensor 404, the second sensor, 406 and the third sensor 408. The emitter 402 thus determines in step 9.10 whether to proceed back to step 9.2 and to remain available to detection energetic pulses, or from step 9.10 to step 9.12 and to perform alternate operations.

It is understood that one or more sensors 404, 406, 408, and sensing elements 113, 114, 115, 302, 303, and 304 may be located, affixed or positioned in various alternate applications of the present invention within the body 410, the heart 102, external to organic tissue, or upon, within or in relation to an inorganic material, a inorganic structure or a robotic element. More particularly, one or more of the first sensor 404, the second sensor, 406 and the third sensor 408, and sensing elements 113, 114, 115, 320, 303, and 304 may be located, affixed or positioned in various alternate applications of aspects of the present invention to or proximate to a heart wall location, a heart chamber wall, a ventricular heart chamber wall, or a heart septal chamber wall.

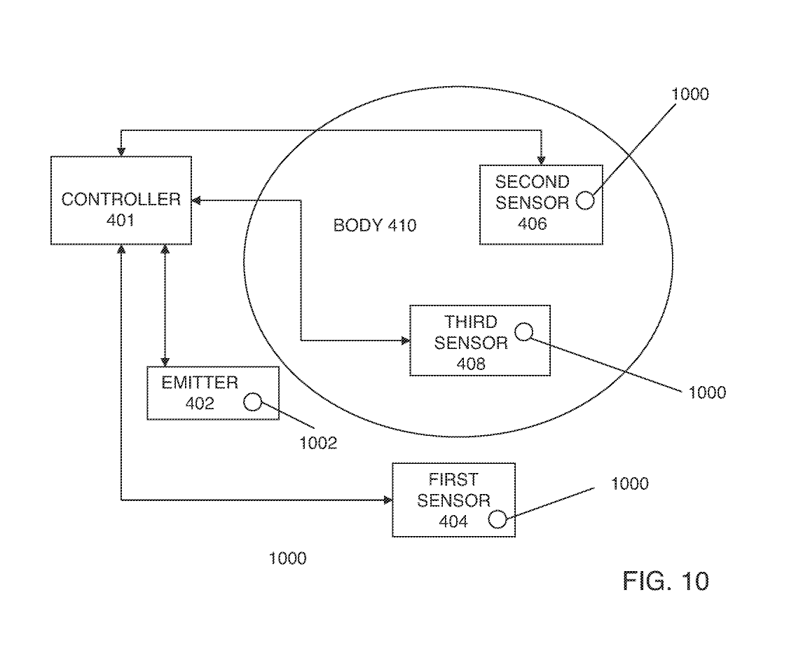

Referring now generally to the Figures and particularly to FIG. 10, FIG. 10 is a schematic drawing of a third device, wherein the emitter 402 and the first sensor 404 are positioned external to the body 410. It is understood that configuration of the emitter 402, the first sensor 404, the second sensor 406 and the third sensor 408 may additionally include an optical energy sensor 1000 configured to detect photonic energy pulses transmitted from a photonic pulse generator 1002 of the emitter 402. It is further understood that configuration of the emitter 402, the first sensor 404, the second sensor 406 and the third sensor 408 may be similar in that the emitter 402 may be additionally configured with a photonic pulse sensor 1000 to detect photonic energy pulses, and that the first sensor 404, the second sensor 406 and/or the third sensor 410 may be additionally configured with a photonic pulse generator 1002.

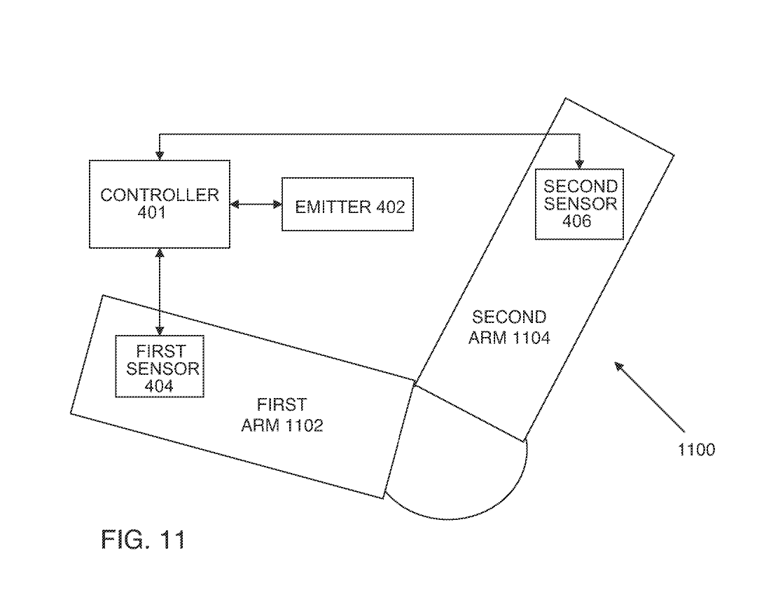

Referring now generally to the Figures and particularly to FIG. 11, FIG. 11 is a schematic drawing of a device, wherein the emitter 402 and the controller 401 are positioned external to a robotic structure 1100. The robotic structure 1100 includes a first arm 1102 which may be movable in relation to a second arm 1104. The first sensor 404 is affixed to the first arm 1102 and the second sensor 406 is affixed to the second arm 1104. The controller 401 determines the instantaneous position of the emitter 402 to both the first sensor 404 and the second sensor 406.

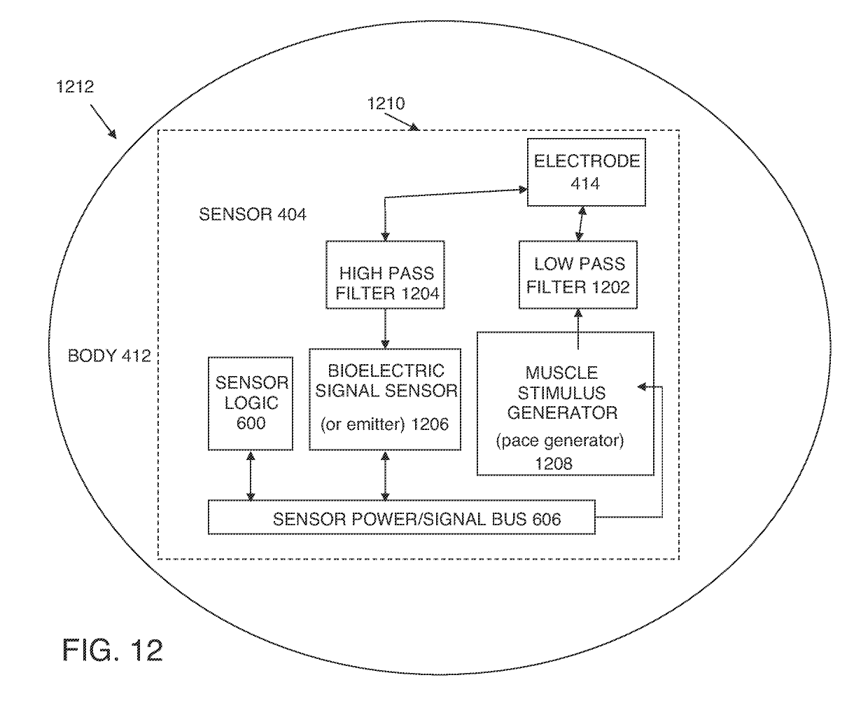

Referring now generally to the Figures and particularly to FIG. 12, FIG. 12 is a schematic diagram of a variation of the first sensor 404, wherein the first sensor 404 comprises a low pass frequency filter 1202, a high pass frequency filter 1204 and a bioelectric signal measurement device (or emitter) 1206. The bioelectric signal sensor measurement device 1206 (hereinafter “bioelectric signal sensor” 1206) and a bioelectric muscle stimulus generator (pace generator) 1208 comprising the cardiac electrode 414 share a same signal pathway 1210 used to send and receive electrical, electromagnetic, photonic and/or acoustic signals to and/or from a bioelectric signal source 1212, such as the heart 102 or the body 412. The first sensor 404 is positioned within the human, mammalian, or animal body 412 and preferably in direct contact with the heart 102 of FIG. 1. The cardiac electrode 414 is configured to generate a cardiac pacing pulse to encourage the heart 102 to contract in a desired manner. The bioelectric signal sensor 1206 is configured to monitor electromagnetic and bioelectric signals, states and activity of, or related to, the body 412 and report detected bioelectrical electromagnetic and bioelectric signals to the controller 401 and/or the pacemaker 412 via the sensor power/signal bus 606. This may act, for example, as a relatively high or a relatively low frequency source or sink. In the prior art, the transmission of a cardiac pacing signal from a cardiac electrode of the prior art can impair the ability of a sensor of the prior to monitor the bioelectric and electromagnetic signals, states and activity of the body 412 by overloading the circuitry of such a sensor. This temporary period of overload of the prior art sensor proximate to, during, and following the generation and delivery of a cardiac pacing pulse that occurs in the prior art is especially undesirable because potentially clinically-significant bioelectric and/or electromagnetic information, though available for capture by during, proximate to, and following the delivery of a cardiac pacing pulse to the heart, may not be sensed or captured by various technologies of the prior art due to the overload condition.

FIG. 12 illustrates a novel and non-obvious configuration of the first sensor 404 that reduces the overloading effect of the generation of the cardiac pacing pulse by cardiac electrode 414 upon the bioelectric signal sensor 1206. As a cardiac pacing pulse travels from the cardiac electrode 414, the cardiac pacing pulse passes through the low pass frequency filter 1202 via the signal pathway 1208 and then onto the heart 102 (FIG. 1). The low pass frequency filter 1202, or “low pass filter” 1202, may comprise an inductor, or other suitable circuitry well known in the art that filters out, eliminates or reduces the amplitude of high frequency components of a cardiac pacing pulse. One millihenry of inductance is a representative inductance value of an inductor element of the low pass filter 1202, which may be, for example, 2.2 MHz cut off. The low pass filter 1202 thereby reduces the amplitude of high frequency electrical components of the cardiac pacing pulse that are available for detection by the bioelectric signal sensor 1206, and the overload effect of the cardiac pacing pulse on the bioelectric signal sensor 1206 is thereby reduced. In addition, a high pass filter is further disposed along the signal pathway 1210 between the output of the low pass filter 1202 and the input of the bioelectric signal sensor 1206. The high pass frequency filter 1204, or “high pass filter” 1204, may comprise a capacitive element, or other suitable circuitry well in the art that filters out, eliminates or reduces the amplitude of low frequency components of a cardiac pacing pulse. 330 picofarads is a representative capacitance value of a capacitive element of the high pass filter 1204, which may be, for example, 100 pF.

The effect of the cardiac pacing pulse upon the bioelectric signal sensor 1206 is thus reduced by disposing the low pass filter 1202 and the high pass filter 1204 between the output of the cardiac electrode 414 and the bioelectric signal sensor 1206. The bioelectric signal sensor 1206 may also generate high frequency signals. The low pass filter 1202 prevents these circuits from loading these high frequency signals, e.g., with reference to electric tomography (ET) as currently practiced.

Computer-Readable Medium

One or more aspects of the subject invention may be in the form of computer-readable medium 418 having programming stored thereon for implementing the subject methods. The computer-readable media 418 may be, for example, in the form of a computer disk or CD, a floppy disc, a magnetic “hard card”, a server, or any other computer-readable media 418 capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer-readable medium 418 may include stored programming embodying an algorithm for carrying out the subject methods. Accordingly, such a stored algorithm is configured to, or is otherwise capable of, practicing the subject methods. The subject algorithm and associated processor may also be capable of implementing the appropriate adjustment(s).

The term “computer-readable medium” as used herein refers to any suitable medium known in the art that participates in providing instructions to the network for execution. Such a medium may take many forms, including but not limited to, non-volatile media, and volatile media. Non-volatile media includes, for example, optical or magnetic disks, tapes and thumb drives. Volatile media includes dynamic memory.

Arrhythmia detection is another area of application of the first method. Current arrhythmia detection circuits rely on electrical activity within the heart 102. Such algorithms are therefore susceptible to confusing electrical noise for an arrhythmia. There is also the potential for misidentifying or mischaracterizing arrhythmia based on electrical events when mechanical analysis would reveal a different underlying physiologic process. Therefore the first method could also be adapted to develop a superior arrhythmia detection and categorization algorithm.

In certain aspects, one or more of the following may be observed:
Low power consumption;
Real time discrimination of multiple lines of position possible (one or more); and
Noise tolerance, since the indicators are relative and mainly of interest in the time domain. Amplitudes in one cardiac cycle vs. other cardiac cycles are of less interest than the time course of different wall segments and their motions relative to each other over a single cardiac cycle. This could convey significant advantages in terms of ability to resist drift and noise caused by changing physiologic conditions or changes in underlying electronics or devices. One example being changes in the pulmonary congestion of the lungs which may over time, alter the amplitude of the received signal at a catheter relative to the emitting electrodes at any given moment.

An important application for the first method of the present invention is to aid the clinician in optimizing resynchronization therapy. The above examples are addressed to that particular application. However, there are numerous other applications for the inventive system and information production process.

Non-cardiac applications will be readily apparent to the skilled artisan, such as, by example, measuring the congestion in the lungs, determining how much fluid is in the brain, assessing distention of the urinary bladder. Other applications also include assessing variable characteristics of many organs of the body such as the stomach. In that case, after someone has taken a meal, the present invention allows measurement of the stomach to determine that ingestion has occurred. Because of the inherently numeric nature of the data from the present invention, these patients can be automatically stimulated to stop eating, in the case of overeating, or encouraged to eat, in the case of anorexia. The present inventive system can also be employed to measure the fluid fill of a patient's legs to assess edema, or other various clinical applications.

While the present invention has been described with reference to the specific applications thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The foregoing disclosures and statements are illustrative only of the present invention, and are not intended to limit or define the scope of the present invention. The above description is intended to be illustrative, and not restrictive. Although the examples given include many specificities, they are intended as illustrative of only certain possible applications of the present invention. The examples given should only be interpreted as illustrations of some of the applications of the present invention, and the full scope of the Present invention should be determined by the appended claims and their legal equivalents. Those skilled in the art will appreciate that various adaptations and modifications of the just-described applications can be configured without departing from the scope and spirit of the present invention. Therefore, it is to be understood that the present invention may be practiced other than as specifically described herein. The scope of the present invention as disclosed and claimed should, therefore, be determined with reference to the knowledge of one skilled in the art and in light of the disclosures presented above.

What is claimed is:

1. A distance measurement system comprising:
an emitter located within a living being and configured to generate an energetic pulse;
a sensor located within the living being and configured to detect the energetic pulse;
a timing circuit coupled with the sensor and configured to determine a time of detection of the energetic pulse by the sensor; and
a logic circuit coupled with the timing circuit and the emitter, and the logic circuit configured to determine a travel time of the energetic pulse measured between a generation of the energetic pulse by the emitter and the time of detection of the energetic pulse by the sensor, and to derive the distance of separation of the emitter and the sensor from the travel time.

2. The distance measurement system of claim 1, wherein the sensor is associated with a human heart.

3. The distance measurement system of claim 2, wherein the emitter is associated with the human heart.

4. The distance measurement system of claim 1, wherein the energetic pulse is selected from the group essentially consisting of an electromagnetic energy pulse and an acoustic energy pulse.

5. The distance measurement system of claim 1, wherein the emitter comprises:
a second pulse emitter circuit configured to generate an alternate energetic pulse, the sensor further configured to detect the alternate energetic pulse, the timing circuit further configured to determine a delay time measured between the detection of the energetic pulse by the sensor and a detection of the alternate energetic pulse by the sensor, and the logic circuit further configured to derive the distance of separation of the emitter and the sensor from the delay time.

6. The distance measurement system of claim 5, wherein the energetic pulse is an electromagnetic energy pulse and the alternate energetic pulse is an acoustic energy pulse.

7. The distance measurement system of claim 1, further comprising:
the sensor comprising a timing circuit, an energetic pulse sensing circuit, a bioelectric signal measurement device, a bioelectric muscle stimulus generator, and a bioelectric signal pathway;
the timing circuit coupled with the energetic pulse sensing circuit and configured to determine a time of detection of the energetic pulse by the energetic pulse sensing circuit;
the logic circuit coupled with the timing circuit and the emitter, and the logic circuit configured to determine a travel time of the energetic pulse measured between a generation of the energetic pulse by the emitter and the time of detection of the energetic pulse by the sensor, and to derive the distance of separation of the emitter and the sensor from the travel time;
the bioelectric signal pathway comprising a high pass filter and a low pass filter;
the high pass filter having a high pass input gate and a high pass output gate, the high pass output gate coupled with the bioelectric signal measurement device;
the low pass filter having a low pass input gate and a low pass output gate, the low pass output gate coupled with the high pass input gate; and
the bioelectric muscle stimulus generator coupled with the low pass input gate, whereby a bioelectric signal source is coupled with the high pass input gate and the low pass output gate, and the high pass filter and the low pass filter protect the bioelectric signal measurement device from interference from the bioelectric muscle stimulus generator.

8. The distance measurement system of claim 1, further comprising:
a second sensor positioned within the living being, the second sensor configured to detect the energetic pulse;
a second timing circuit coupled with the logic circuit and the second sensor and configured to determine a second time of detection of the energetic pulse by the second sensor; and
the logic circuit further configured to determine a second travel time of the energetic pulse measured between the generation of the energetic pulse by the emitter and the second time of detection of the energetic pulse by the second sensor, and to derive a second distance of separation of the emitter and the second sensor from the second travel time.

9. The distance measurement system of claim 8, further comprising:
a third sensor positioned within the living being, the third sensor configured to detect the energetic pulse;
a third timing circuit coupled with the logic circuit and the third sensor and configured to determine a third time of detection of the energetic pulse by the third sensor; and
the logic circuit further configured to determine a travel time of the energetic pulse measured between the generation of the energetic pulse by the emitter and the third time of detection of the energetic pulse by the third sensor, and to derive a third distance of separation of the emitter and the third sensor from the third travel time.

10. A method comprising:
locating an energetic pulse emitter within a living being;
locating a sensor within the living being, the sensor configured to detect an energetic pulse;
generating an energetic pulse from the energetic pulse emitter;
determining a travel time of the energetic pulse between the energetic pulse emitter and the sensor; and
deriving a distance between the energetic pulse emitter and the sensor from the travel time.

11. The method of claim 10, wherein the living being includes a heart organ and the method further comprises coupling the energetic pulse emitter and the sensor to the heart organ.

12. The method of claim 11, further comprising:
locating a second sensor within the living being, the second sensor configured to detect the energetic pulse;
determining a second travel time of the energetic pulse between the energetic pulse emitter and the second sensor; and
deriving a second distance between the energetic pulse emitter and the second sensor from the second travel time.

13. The method of claim 12, further comprising:
locating a third sensor within the living being, the second sensor configured to detect the energetic pulse;
determining a third travel time of the energetic pulse between the energetic pulse emitter and the third sensor; and
deriving a third distance between the energetic pulse emitter and the third sensor from the third travel time.

14. The method of claim 10, further comprising:
generating an alternate energetic pulse from the energetic pulse emitter;
determining a delay time measured between a detection of the energetic pulse by the sensor and a detection of the alternate energetic pulse by the sensor; and
deriving a distance between the energetic pulse emitter and the sensor from the delay time.

15. The method of claim 14, wherein the energetic pulse is an electromagnetic energy pulse and the alternate energy pulse is an acoustic energy pulse.

16. A device comprising:
a bioelectric signal measurement device;
a high pass filter having a high pass input gate and a high pass output gate, the high pass output gate coupled with the bioelectric signal measurement device;
a low pass filter having a low pass input gate and a low pass output gate, the low pass output gate coupled with the high pass input gate; and
a bioelectric muscle stimulus generator coupled with the low pass input gate; and
a bioelectric signal source coupled with the high pass input gate and the low pass output gate.

17. The device of claim 16, wherein the high pass filter comprises a capacitor.

18. The device of claim 16, wherein the low pass filter comprises an inductor.

19. The device of claim 16, wherein the bioelectric signal measurement device is further configured to enable detection of bioelectric signals of the living being within a range of one picosecond to one millisecond after a transmission a trailing edge of the energetic pulse.

20. The device of claim 19, wherein the range is a range of one microsecond to ten microseconds.
